(19) United States
(12) Patent Application Publication
(10) Pub. No.: US 2011/0130471 A1
(43) Pub. Date: Jun. 2, 2011

(54) POLYURETHANES

(75) Inventors: Cindy Münzenberg, Duesseldorf (DE); Oliver Pietsch, Mulheim (DE); Anja Wick, Hilden (DE); Eike Ulf Mahnke, Velbert (DE); Paul Birnbrich, Solingen (DE)

(73) Assignee: Cognis IP Management GmbH, Duesseldorf (DE)

(21) Appl. No.: 12/950,663

(22) Filed: Nov. 19, 2010

(30) Foreign Application Priority Data
Nov. 27, 2009 (EP) 09014771.1

(51) Int. Cl. A61K 8/87; C08G 18/75; C08L 75/04; C08K 5/06

(52) U.S. Cl. 514/772.3; 528/76; 524/591; 524/376

(57) ABSTRACT

Polyurethanes, suitable as thickeners for aqueous systems, are obtained by reacting (i) one or more hydrophilic polyols containing at least two OH groups and at least two functional groups selected from ether and ester groups, having a molecular weight of at least 300 Daltons; (ii) one or more C8 to C36 Guerbet alcohols, which are produced from monoalcohols having 4 to 18 carbon atoms selected from the group of primary and/or secondary monoalcohols of formula (MA),

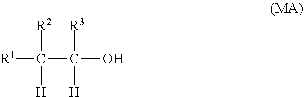

where (a) the total number of carbon atoms in compounds (MA) is 4 to 18; (b) R¹, R² and R³ are independently hydrogen, or linear, branched or alicyclic, saturated or unsaturated alkyl; (c) optionally, R¹ and R², and/or R¹ and R³, and/or R² and R³ are linked together to form an alicyclic substructure; and (d) compounds (MA) have at least one branch; and (iii) one or more at least difunctional isocyanates.

### CROSS-REFERENCE TO RELATED APPLICATIONS

This application claims the benefit of priority under 35 USC §119 of European Patent Application number 09014771.1, filed on Nov. 27, 2009, which is incorporated herein in its entirety by reference.

### FIELD OF THE INVENTION

The invention relates to polyurethanes with a specific structure, and thickener concentrates comprising these polyurethanes.

### BACKGROUND OF THE INVENTION

Polyurethane solutions or dispersions in water-dilutable aqueous or predominantly aqueous phase are referred to by the person skilled in the art as HEUR thickeners (the acronym HEUR is derived from “nonionic hydrophobically modified ethylene oxide urethane block copolymer”) and have been used for a relatively long time in highly diverse fields of application for thickening water-based emulsion paints.

The thickeners of the HEUR type described as early as the end of the 1970s in U.S. Pat. No. 4,079,028 are composed of linear and/or branched polyethylene glycol blocks and hydrophobic segments which are generally linked together via urethane groups (when using amines instead of alcohols, urea groups result).

The principle of action for the thickening effect of the HEUR thickeners is assumed to be that the polyethylene glycol segments ensure the water compatibility and the hydrophobic segments construct a viscosity-imparting three-dimensional molecular association via an association with one another and also with dispersed binder particles of the emulsion paint to be thickened therein.

Preferred hydrophobic building blocks in standard commercial HEUR thickeners are relatively long-chain, generally monofunctional alcohols, such as, for example, n-octanol, n-dodecanol, isotridecyl alcohol, isononylphenol or methyl ricinoleate. These alcohols are used predominantly as they are, but also in the form of their addition products with a few equivalents of ethylene oxide. The polyfunctional isocyanate building blocks used predominantly in standard commercial HEUR thickeners are generally difunctional. For example, methylenebis(4-cyclohexyl)diisocyanate, m/p-tetramethylenexylylene diisocyanate, hexamethylene diisocyanate, 2,4-tolylene diisocyanate, trimethylhexamethylene diisocyanate or 4/2,4′-diphenylmethane diisocyanate are used. The polyethylene glycol building blocks used in standard commercial HEUR thickeners are generally likewise difunctional and have molecular weights in the region of a few thousand daltons, for example 4500 or 10 000 daltons.

The use ratios of the individual building blocks of HEUR thickeners—whether branched or unbranched polyethylene glycols, mono- or polyfunctional hydrophobic alcohols, preethoxylated mono- or polyfunctional hydrophobic alcohols, di- or polyfunctional short-chain alcohols used as chain extenders—are generally selected such that in each case one hydrophobic alcohol is in each case still available to each one via an ethylene glycol segment end reactive with hydroxyl groups.

The hydroxyl-terminated synthesis building blocks of HEUR thickeners are joined together through reaction with di- or polyfunctional isocyanates, where the equivalent use ratios of the isocyanate groups and of the “H-acidic” groups (generally OH groups, although NH₂ groups are also possible) to be brought together for the addition reaction are selected such that at least slightly less than one isocyanate group equivalent is facing each “H-acidic” group equivalent, thus as a rule each OH group. In other words: the OH:NCO equivalent ratio is generally adjusted to a value of at least 1:1, where ideally 1:1 is aimed for or the OH groups predominate compared to the NCO groups by 5-10% (which corresponds to an equivalent ratio of OH:NCO in the range from 1.05:1 to 1.1:1) in order to ensure that the end product (the HEUR thickener) contains no free NCO groups, which are undesired on the one hand for toxicological reasons, and on the other hand because, upon subsequent use in formulations to be thickened, they can enter into undesired secondary reactions with formulation constituents. This basic principle that namely during the production of HEUR thickeners the OH groups of the polyethylene glycol building blocks and hydrophobic alcohol building blocks predominate slightly by ca. 5-10% compared to the NCO groups of the isocyanate building blocks is already part of the teaching of the aforementioned U.S. Pat. No. 4,079,028 (cf. therein column 3, line 17ff).

EP-B-905175 describes specific polyurethane thickeners which comprise isocyanates, polyetherpolyols and capping agents as building blocks, the latter having a quite specific structure described more precisely by a formula. This structure is characterized in that the capping agent is a monoalcohol, the structure of which is branched at a single carbon atom. Section [0040] lists in particular the ISOFOL® range of branched alcohols from Condia as an example of suitable capping agents. As the person skilled in the art is aware, these are so-called Guerbet alcohols, which are obtainable via dimerization of linear alcohols. In structural terms, these Guerbet alcohols have a single branch per molecule.

Guerbet alcohols are special branched alcohols. They are primary alcohols which are branched in the beta position relative to the CH₂OH group. Guerbet alcohols are known to the person skilled in the art, and some have been commercially available for a long time. They are obtained by the so-called Guerbet reaction, a dimerization reaction known for about 100 years, which can be described by the following reaction scheme (R* signifies an aliphatic group therein):

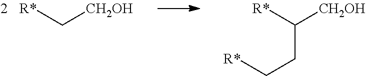

In the course of the classic Guerbet reaction, a primary or secondary alcohol is converted to a primary alcohol of approximately twice the molecular weight, which is alkylated in the beta position relative to the carbon atom which carries the OH group. Thus, for example, n-butanol is converted to 2-ethylhexan-1-ol, hexan-1-ol is converted to 2-butyloctan-1-ol and octan-1-ol is converted to 2-hexyldodecan-1-ol.

The primary or secondary alcohols used for the Guerbet reaction carry at least one hydrogen atom on the carbon atom which is directly adjacent to the carbon atom with the OH group—in many cases, they carry two hydrogen atoms, i.e. a methylene group is then directly adjacent to the carbon atom with the OH group.

The Guerbet reaction typically proceeds in the presence of a base at elevated temperature with the elimination of water and constitutes a simple option for converting linear alcohols to branched alcohols. Typically, only one single alcohol is used in the Guerbet reaction. However, it is also possible to use two different alcohols; in this case, one speaks of a mixed Guerbet reaction.

In the above formula scheme, the radicals R* are in each case an aliphatic group. Typically, this is a linear and saturated aliphatic group, i.e. for example fatty alcohols such as octanol or decanol are used as starting alcohols for the Guerbet reaction. Sometimes, R* is a saturated cycloaliphatic group, i.e. for example alcohols such as cyclopentanol or cyclohexanol are used as starting alcohols for the Guerbet reaction.

### DETAILED DESCRIPTION OF THE INVENTION

It was an object of the invention to provide thickeners based on polyurethane which, compared with the polyurethane thickeners from the prior art, achieve an increased viscosity of the thickened product coupled with a comparatively lower intrinsic viscosity of the thickeners in their formulation form in the same amount. Moreover, it should be possible to prepare the thickeners, if so desired, without using volatile organic solvents.

The present invention firstly provides polyurethanes obtainable by reacting
(i) one or more hydrophilic polyols which contain, per molecule, at least two OH groups and at least two functional groups which are selected from the functions —O— (ether groups) and —COO— (ester groups), where the molecular weight of these hydrophilic compounds is at least 300 daltons;
(ii) one or more monoalcohols having in total 8 to 36 carbon atoms, with the proviso that these mono-alcohols are Guerbet alcohols (GA) which are obtained by using as starting alcohols for the Guerbet reaction for producing these Guerbet alcohols (GA) exclusively those monoalcohols having 4 to 18 carbon atoms which are selected from the group of primary and/or secondary monoalcohols of the formula (MA),

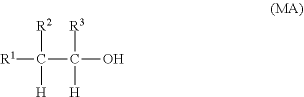

where: (a) the total number of carbon atoms in the compounds (MA) is in the range from 4 to 18; (b) the radicals R¹, R² and R³ are hydrogen or alkyl groups which—independently of one another—may be linear or branched or alicyclic, saturated or unsaturated; (c) optionally the radicals R¹ and R² and/or R¹ and R³ and/or R² and R³ can be linked together, i.e. be part of an alicyclic substructure; and (d) the compounds (MA) have at least one branch; and
(iii) one or more at least difunctional isocyanates.

### The Polyols (i)

The hydrophilic polyols (i) contain per definitionem per molecule at least two OH groups and at least two functional groups which are selected from the functions —O— (ether groups) and —COO— (ester groups), where the molecular weight of these hydrophilic compounds is at least 300, and preferably at least 1000 daltons. The polyols (i) are therefore the hydrophilic molecular building block of the polyurethanes according to the invention of the HEUR type. Reference may be expressly made to the fact that the compounds (i) differ fundamentally from those compounds (ii), which are not hydrophilic but hydrophobic.

Of suitability as compounds (i) are, for example, the polymerization products of ethylene oxide, the mixed or graft polymerization products thereof and also the products obtained by condensation of polyhydric alcohols or mixtures thereof and the polyethers obtained by ethoxylation of polyhydric alcohols, amides, polyamides and aminoalcohols. Examples of suitable compounds (i) are for example polyethylene glycols, addition products of ethylene oxide onto trimethylolpropane, EO-PO block copolymers, OH-terminated polyesters, such as, for example, those of the type of polyfunctional polycaprolactones.

Preferred compounds (i) are polyetherpolyols. These are those hydrophilic polyols (i) which contain at least two OH groups and at least two —O— functions (ether groups) per molecule. These polyetherpolyols are generally so hydrophilic that they are water-soluble.

Of suitability for producing the polyurethanes according to the invention are preferably those polyetherpolyols which contain at least predominantly polyethylene glycol. Particularly good results are achieved if these polyethylene glycols have an average content of alkoxy units in the range from 20 to 400. As compounds (i), preference is given to diols of the general formula HO—(CH₂—CH₂—O)ₓ—H, where x can assume the values 30 to 300. These are polyethylene glycols which are condensation products of ethylene oxide with ethylene glycol or water. Preferably, the molecular weight of these polyethylene glycols is adjusted to values in the range from 2000 to 20 000 daltons. Polyethylene glycols with a molecular weight in the range from 4000 to 10 000 daltons are particularly preferred as compounds (i).

### The Monoalcohols (ii)

Component (i) comprises monoalcohols having in total 8 to 36 carbon atoms, with the proviso that these monoalcohols are Guerbet alcohols (GA) obtained by using as starting alcohols for the Guerbet reaction for producing these Guerbet alcohols (GA) exclusively those monoalcohols having 4 to 18 carbon atoms which are selected from the group of primary and/or secondary monoalcohols of the formula (MA),

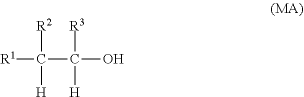

where: (a) the total number of carbon atoms in the compounds (MA) is in the range from 4 to 18; (b) the radicals R¹, R² and R³ are hydrogen or alkyl groups which—independently of one another—may be linear or branched or alicyclic, saturated or unsaturated; (c) optionally the radicals R¹ and R² and/or R¹ and R³ and/or R² and R³ can be linked together, i.e. be part of an alicyclic substructure; (d) the compounds (MA) have at least one branch.

Guerbet alcohols, and the Guerbet reaction used for producing them, are known to the person skilled in the art, with important points on this topic already having been explained in the section relating to the prior art (see above). As is evident from the formula scheme shown in this connection (see above), the Guerbet reaction ultimately consists in linking together two molecules of starting alcohol in a condensation reaction, where the resulting molecule, i.e. the Guerbet alcohol, contains a branch in the molecule at the point where the two original starting alcohol building blocks are linked together. If R* in the above formula scheme is a linear aliphatic group or a cycloaliphatic group, the resulting Guerbet alcohol contains only one branch in the molecule. If, however, R* in the above formula scheme is a group which contains a branch, then the resulting Guerbet alcohol contains three branches in the molecule, namely one which results from the linkage reaction (the Guerbet reaction) and two which originate from the starting alcohols used. It is essential for the understanding of the present invention that the monoalcohols (MA) must have at least one branch; this is unequivocally expressed by the above condition (d).

Preferably, the alkyl groups of the compounds (MA) here are exclusively saturated.

In the case of the compounds (MA) whose alkyl radicals are exclusively saturated, in a preferred embodiment it is the case that the total number of their carbon atoms is in the range from 5 to 10. In this connection, alcohols having 5 carbon atoms and in particular isomer mixtures of alcohols having 5 carbon atoms are particularly preferred.

3-Methylbutan-1-ol is very particularly preferred as monoalcohol (MA). It can be used as starting alcohol in pure form or in the form of technical-grade mixtures and also in a mixture with other alcohols of the formula (MA). As a result of a Guerbet reaction, when using 3-methylbutan-1-ol, the monoalcohol (MA) formed is the following Guerbet alcohol (GA*)

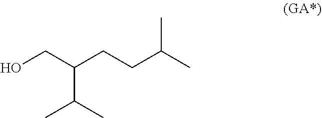

which can be referred to as 2-isopropyl-5-methylhexan-1-ol.

The compounds (MA) can be used in pure form or in the form of technical-grade products. It is also possible, for the Guerbet reaction to be carried out according to the invention, to use mixtures which essentially comprise one or more compounds (MA) besides further substances, where unbranched aliphatic alcohols are excluded as further substances.

As is known, hydrogen bonded to N, O or S is then referred to as Zerewitinoff-active hydrogen (sometimes also only as “active hydrogen”) if it produces methane according to a process discovered by Zerewitinoff through reaction with methylmagnesium iodide. Since the compounds (ii) are Guerbet alcohols (GA) and therefore contain an OH group, then they also contain Zerewitinoff-active hydrogen precisely on account of this OH group.

### The Isocyanates (iii)

Suitable at least difunctional isocyanates (iii) are all polyfunctional aromatic, alicyclic and aliphatic isocyanates. Preferably, the suitable polyfunctional isocyanates comprise on average 2 to at most 4 NCO groups. Diisocyanates are preferred as compounds (iii).

For example, suitable isocyanates which may be mentioned are 1,5-naphthalene diisocyanate, 4,4′-diphenylmethane diisocyanate (MDI), hydrogenated MDI (H₁₂MDI), xylylene diisocyanate (XDI), tetramethylxylene diisocyanate (TMXDI), 4,4′-diphenyldimethylmethane diisocyanate, di- and tetraalkyldiphenylmethane diisocyanate, 4,4′-dibenzyl diisocyanate, 1,3-phenylene diisocyanate, 1,4-phenylene diisocyanate, the isomers of tolylene diisocyanate (TDI), optionally in a mixture, 1-methyl-2,4-diioscyanatocyclohexane, 1,6-diisocyanato-2,2,4-trimethylhexane, 1,6-diisocyanato-2,4,4-trimethylhexane, 1-isocyanatomethyl-3-isocyanato-1,5,5-trimethylcyclohexane, chlorinated and brominated diisocyanates, phosphorus-containing diisocyanates, 4,4′-diisocyanatophenylperfluoroethane, tetramethoxybutane 1,4-diisocyanate, butane 1,4-diisocyanate, hexane 1,6-diisocyanate (HDI), dicyclohexylmethane diisocyanate, cyclohexane 1,4-diisocyanate, ethylene diisocyanate, bis-isocyanatoethyl phthalate, also polyisocyanates with reactive halogen atoms, such as 1-chloromethylphenyl 2,4-diisocyanate, 1-bromomethylphenyl 2,6-diisocyanate, 3,3-bischloromethyl ether 4,4′-diphenyldiisocyanate. Sulfur-containing polyisocyanates are obtained for example by reacting 2 mol of hexamethylene diisocyanate with 1 mol of thiodiglycol or dihydroxydihexyl sulfide. Further important diisocyanates are trimethylhexamethylene diisocyanate, 1,4-diisocyanatobutane, 1,2-diisocyanatododecane and dimer fatty acid diisocyanate. Worthy of interest are in part capped polyisocyanates which permit the formation of self-crosslinking polyurethanes, e.g. dimeric tolylene diisocyanate, or polyisocyanates partially reacted with, for example, phenols, tertiary butanol, phthalimide, caprolactam.

According to the invention, it is preferred that the isocyanates (iii) used for producing the polyurethanes comprise at least predominantly isophorone diisocyanate (IPDI) and/or tetramethylxylene diisocyanate (TMXDI). Component (iii) is preferably exclusively selected from the group consisting of isophorone diisocyanate (IPDI) and tetramethylxylene diisocyanate (TMXDI).

In one preferred embodiment, isocyanates with a functionality of 2 (difunctional isocyanates) are used.

In another embodiment, isocyanates with a functionality above 2 are used—proportionately or entirely—if the desire is to produce polyurethanes with a branched structure.

### Special Equivalent Ratios

In one embodiment, it is the case that the compounds (i), (ii) and (iii) are reacted with one another in the equivalent ratios OHᵢ₎:ZHᵢᵢ₎:NCOᵢᵢᵢ₎ of 1:(1+x):2(1+y), with the proviso that the following conditions apply:
x is a number in the range from 0.05 to 1.2 and
y is a number in the range from (0.2 to 1.05)x, and
equivalent ratio NCOᵢᵢᵢ₎>(OHᵢ₎+ZHᵢᵢ₎)

The expression OHᵢ₎ refers to the primary (terminal) OH groups of the compounds (i). The expression ZHᵢᵢ₎ refers to the functional groups with Zerewitinoff-active hydrogen atom reactive towards NCO groups (ii). The expression NCOᵢᵢᵢ₎ refers to the isocyanate groups of the compounds (iii).

Within the context of the present invention, the equivalents of the compounds (i) are OH equivalents, the compounds (ii) are Zerewitinoff-active hydrogen equivalents and the compounds (iii) are NCO equivalents.

Although the concept of equivalent is familiar to the person skilled in the art in the field of polyurethane chemistry under discussion here, for the sake of clarity, it will be described below what is meant by this.

The expression “equivalents” is to be understood in the customary sense, and focuses on the available reactive groups of molecules. Thus, for example, 1 mol of a monoalcohol contains 1 mol of OH groups; 1 mol of a diol contains 2 mol of OH groups, 1 mol of a triol contains 3 mol of OH groups, etc. Quite analogously, 1 mol of a diisocyanate (NCO functionality=2) contains 2 mol of NCO groups, 1 mol of a polyisocyanate mixture with an (average) functionality of 2.3 contains on average 2.3 mol of NCO groups, etc. If, for example, the wish is to react alcohols and isocyanates together such that the compounds used should be in certain ratios, based on the OH or NCO groups, respectively, then it is recommended to use the ratios of the reactive groups instead of weight ratios or molar ratios. This OH:NCO ratio is referred to as the equivalent ratio.

Expressed in general terms, the equivalent ratio is the numerical ratio of defined reactive groups in the reactants used.

For illustrative purposes, it may additionally be explained by means of a practical example how an equivalent ratio is ascertained in a simple manner. If, for example, within the context of an embodiment of the teaching according to the invention,
1 mol of a polyethylene glycol (i) (PEG, OH functionality=2) with two OH groups per molecule is reacted with
4 mol of a hydrophobic alcohol (ii) (OH functionality=1) with one OH group per molecule and
4 mol of a diisocyanate (iii) (NCO functionality=2) to give the polyurethane, then
the PEG (i) used contains 2 mol of OH groups,
the hydrophobic alcohol (ii) used contains 4 mol of OH groups and
the diisocyanate (iii) used contains 8 mol of NCO groups.

The numerical ratio of the OH groups of the polyethylene glycol to the OH groups of the hydrophobic alcohol to the NCO groups of the diisocyanate is therefore 2:4:8 or 1:2:4.

Or vice versa: if it is the wish to react the components just mentioned (PEG, hydrophobic alcohol and diisocyanate) in an equivalent ratio of 1:3:3, then polyethylene glycol, hydrophobic alcohol and diisocyanate have to be used in a molar ratio of 0.5:3:1.5 or 1:6:3.

Preferably, x is a number in the range from 0.2 to 1.0 and in particular in the range from 0.5 to 1.0.

For the sake of clarity and unambiguity, it may be expressly determined that y arises through multiplication. The expression stated for y, namely “(0.2 to 1.05)x” therefore means that x—for which a number is to be used from the range stated for x—has to be multiplied by a number from the range 0.2 to 1.05.

Preferably, y is a number in the range from (0.4 to 1.0)x and in particular in the range from (0.6 to 1.0)x.

In one preferred embodiment, x is a number in the range from 0.2 to 1.0 and y is a number in the range from (0.4 to 1.0)x.

In a further preferred embodiment, x is a number in the range from 0.5 to 1.0 and y is a number in the range from (0.4 to 1.0)x.

### Optional Polyurethane Building Blocks

As already explained, the building blocks (i), (ii) and (iii) are obligatory for the polyurethanes according to the invention.

In one embodiment, the polyurethanes according to the invention are obtainable by reacting the compounds (i), (ii), (iii) and (iv), where the compounds (iv) are polyols with at least two OH groups per molecule which, apart from the OH groups, contain no further functional groups.

Alcohols with a functionality of from 2 to 4 are particularly suitable as—optional—compounds (iv).

It may be expressly stated that the compounds (iv) are polyfunctional alcohols which, besides the OH groups, contain no further functional groups. In particular, it may be pointed out that there is here no overlap with the polyetherpolyols (i), for example, which do likewise contain a plurality of OH groups, but inevitably additionally —O— groups (ether groups). Consequently, the compounds (iv) are different from the compounds (i).

In one embodiment, alcohols with a functionality of 2 are used as compounds (iv), i.e. diols, in particular diols with terminal OH groups. If the wish is to bring about branches when building up the polyurethane molecule, then at least trifunctional alcohols are to be used as compounds (iv). Preferably, the polyols (iv) then contain at least predominantly trifunctional alcohols, such as, for example, glycerol. Within the context of the invention, a preferred trifunctional alcohol is trimethylolpropane (TMP) as polyol (iv). However, it is also possible to achieve branches upon building up the polyurethane molecule through the use of isocyanates with a functionality higher than 2. Here, triisocyanates are preferred. To optimize the application properties of the thickeners, it is useful to limit such branching positions within the polyurethane molecule to a certain area.

In one embodiment, the polyurethanes are obtainable by reacting the compounds (i), (ii), (iii), (iv) and (v), where the compounds (v) are monoalcohols which are different from the compounds (ii). The monoalcohols (v) are preferably selected from the group of Guerbet alcohols having 10 to 24 carbon atoms which have at most two branches per molecule, and of the so-called oxo alcohols.

### Thickener Concentrates

Thickener concentrates comprising one or more of the above-described polyurethanes according to the invention, and water. In order to avoid unnecessary repetitions, it may be expressly clarified that “above-described polyurethanes according to the invention” are to be understood as meaning all above-described polyurethanes including their specific embodiments.

In one embodiment, these thickener concentrates additionally comprise one or more organic solvents (L) and/or nonionic surfactants (T) of the type of the addition compounds of ethylene oxide and/or propylene oxide onto fatty alcohols having 4 to 18 carbon atoms.

The solvents (L) are volatile organic solvents. Examples thereof are low molecular weight alcohols such as methanol, ethanol, n-propanol, isopropanol, n-butanol, isobutanol, sec-butanol, ethanediol, propanediol, butanediol, glycerol, trimethylolpropane. Preferred nonionic surfactants (T) of the type of the addition compounds of ethylene oxide and/or propylene oxide onto alcohols having 4-18 carbon atoms are those with 2 to 4 mol of ethylene oxide per mol of alcohol. The carbon backbone of the alcohols here may be saturated or unsaturated, linear or branched. An example of a suitable compound (C) of this class which may be mentioned is for example DEHYDOL® O4 (commercial product from Cognis), an addition product of 4 mol of ethylene oxide onto 1 mol of octanol.

### Use

Furthermore, the invention relates to the use of the polyurethanes according to the invention or of the thickener concentrates according to the invention for thickening aqueous systems, preferably aqueous dispersions, selected from the group consisting of aqueous automotive finishes and industrial coatings, printing and textile inks, pigment printing pastes, aqueous pharmaceutical formulations, cosmetic formulations or pharmaceutical-cosmetic formulations, crop protection formulations, filler and pigment dispersions, preparations of detergents, adhesives, waxes and polishes, and also for petroleum recovery, in particular for thickening aqueous colored renders and paints in dispersion form.

### EXAMPLES

### Abbreviations Used

PEG 8000: PLURIOL® E 8000 (polyethylene glycol from BASF)
IPDI: Isophorone diisocyanate (IPDI, Degussa/Hüls)
C10-V3-OH Tri-branched alcohol having 10 carbon atoms which has been prepared as follows by Guerbet reaction from 3-methyl-1-butanol in the presence of catalytic amounts of aldehyde, alkali and palladium on carbon: a reaction mixture of 2500 g of 3-methyl-1-butanol, 75 g of 3-methyl-1-butanal, 4 g of Pd/C (5% Pd/C 3610 from Johnson Matthey in 50% water) was heated to 180° C., 320 g of KOH (50% strength) were metered in in portions and the mixture was subjected to a pressure ramp from 4.6 to 1.4 bar over 18 hours with stirring.
EXXAL® 11 Isodecyl alcohol (oxomethylated propene trimer; commercial product EXXAL® 10 from Exxon Mobil Chemical)
C10-V2-OH 2-Propylheptanol (Bayer; monobranched alcohol having 10 carbon atoms)
Nonanol 3,5,5-Trimethylhexanol (Kyowa)
DEHYDOL® O4 Addition product of 4 mol of ethylene oxide onto 1 mol of n-octanol (DEHYDOL® O4 deo; Cognis)
Demin. water Demineralized water
NEOCRYL® XK 98 Acrylic emulsion (DSM NeoResins)
DEHYDRAN® 1513 Mixture of polymers, emulsifiers and silicone (Cognis)

### Polyurethane Preparation

### Example 1 (E1)

198 g (24 mmol) of PEG 8000 were introduced as initial charge in the 1 l four-neck flask. It was evacuated twice and aerated with nitrogen. A vacuum was then applied and the mixture was heated to 100° C. At this temperature and under a reduced pressure of at least 10 mbar, water was removed over a period of 2 hours. The system was then aerated with nitrogen and, in the further course, the protective-gas atmosphere was maintained by a gentle stream of nitrogen. The mixture was stirred over the entire following reaction time using a stirrer speed of 120 rpm. Then, in succession, 11.40 g of C10-V3-OH (72 mmol) and 16.0 g of isophorone diisocyanate (72 mmol) were added. The reaction temperature was held at 110° C. during the addition and during the subsequent reaction time. As soon as residual isocyanate could no longer be detected (this was the case after a total reaction time of ca. 60 minutes), the temperature was kept at 110° C. and 134.1 g of DEHYDOL® O4 were added. This mixture was stirred until homogeneous. Then, 317.2 g of demin. water were added with stirring. After the product had cooled down, ca. 652.5 g of a viscous, clear, slightly yellowish polymer solution were isolated from the reaction vessel.

The dry residue (for its determination ca. 2 g of the polymer solution prepared as described above were dried in a 10 cm aluminum dish at 105° C. for 1.5 h in a convection drying cabinet) was 51.3% by weight and the Brookfield viscosity was 5600 mPas (Brookfield RVT viscometer/spindle 3/10 rpm/23° C.). The coating viscosity in the Epprecht viscometer was 220 mPas (spindle C).

### Example 2 (E2)

198 g (24 mmol) of PEG 8000 (polyethylene glycol from BASF) were introduced as initial charge in the 1 l four-neck flask. It was evacuated twice and aerated with nitrogen. A vacuum was then applied and the mixture was heated to 100° C. At this temperature and under a reduced pressure of at least 10 mbar, water was removed over a period of 2 hours. The system was then aerated with nitrogen and, in the further course, the protective-gas atmosphere was maintained by a gentle stream of nitrogen. The mixture was stirred over the entire following reaction time using a stirrer speed of 120 rpm. Then, in succession, 3.3 g of Exxal 11 (19.2 mmol), 8.4 g of C10-V3-OH (52.8 mmol) and 16.0 g of isophorone diisocyanate (72 mmol) were added. The reaction temperature was held at 110° C. during the addition and during the subsequent reaction time. As soon as residual isocyanate could no longer be detected (this was the case after a total reaction time of ca. 45 minutes), the temperature was kept at 110° C. and 134.9 g of DEHYDOL® O4 were added. This mixture was stirred until homogeneous. Then, 319.1 g of demin. water were added with stirring. After the product had cooled down, ca. 660 g of a viscous, clear, slightly yellowish polymer solution were isolated from the reaction vessel.

The dry residue (for its determination ca. 2 g of the polymer solution prepared as described above were dried in a 10 cm aluminum dish at 105° C. for 1.5 h in a convection drying cabinet) was 51.6% by weight and the Brookfield viscosity was 5400 mPas (Brookfield RVT viscometer/spindle 3/10 rpm/22° C.)

The coating viscosity in the Epprecht viscometer was 260 mPas (spindle C).

### Example 3 (E3)

198 g (24 mmol) of PEG 8000 were introduced as initial charge in the 1 l four-neck flask. It was evacuated twice and aerated with nitrogen. A vacuum was then applied and the mixture was heated to 100° C. At this temperature and under a reduced pressure of at least 10 mbar, water was removed over a period of 2 hours. The system was then aerated with nitrogen and, in the further course, the protective-gas atmosphere was maintained by a gentle stream of nitrogen. The mixture was stirred over the entire following reaction time using a stirrer speed of 120 rpm. Then, in succession, 9.5 g of C10-V3-OH (60 mmol) and 16.0 g of isophorone diisocyanate (72 mmol) were added. The reaction temperature was held at 110° C. during the addition and during the subsequent reaction time. As soon as residual isocyanate could no longer be detected (this was the case after a total reaction time of ca. 60 minutes), the temperature was kept at 110° C. and 132.0 g of DEHYDOL® O4 were added. This mixture was stirred until homogeneous. Then, 312.2 g of demin. water were added with stirring. After the product had cooled down, ca. 630 g of a viscous, clear, slightly yellowish polymer solution were isolated from the reaction vessel.

The dry residue (for its determination ca. 2 g of the polymer solution prepared as described above were dried in a 10 cm aluminum dish at 105° C. for 1.5 h in a convection drying cabinet) was 51.1% by weight and the Brookfield viscosity was 8700 mPas (Brookfield RVT viscometer/spindle 3/10 rpm/23° C.)

The coating viscosity in the Epprecht viscometer was 420 mPas (spindle C).

### Comparative Example 1 (C1)

198 g (24 mmol) of PEG 8000 (OH number=13.6) were introduced as initial charge in the 1 l four-neck flask. It was evacuated twice and aerated with nitrogen. A vacuum was then applied and the mixture was heated to 100° C. At this temperature and under a reduced pressure of at least 10 mbar, water was removed over a period of 2 hours. The system was then aerated with nitrogen and, in the further course, the protective-gas atmosphere was maintained by a gentle stream of nitrogen. The mixture was stirred over the entire following reaction time using a stirrer speed of 120 rpm. Then, in succession, 11.40 g of C10-V2-OH (72 mmol) and 16.0 g of isophorone diisocyanate (72 mmol) were added. The reaction temperature was held at 110° C. during the addition and during the subsequent reaction time. As soon as residual isocyanate could no longer be detected (this was the case after a total reaction time of ca. 60 minutes), the temperature was kept at 110° C. and 132.8 g of DEHYDOL® O4 were added. This mixture was stirred until homogeneous. Then, 314.0 g of demin. water were added with stirring. After the product had cooled down, ca. 643.0 g of a viscous, clear, slightly yellowish polymer solution were isolated from the reaction vessel.

The dry residue (for its determination ca. 2 g of the polymer solution prepared as described above were dried in a 10 cm aluminum dish at 105° C. for 1.5 h in a convection drying cabinet) was 50.9% by weight and the Brookfield viscosity was 4850 mPas (Brookfield RVT viscometer/spindle 3/10 rpm/23° C.)

The coating viscosity in the Epprecht viscometer was 200 mPas (spindle C).

### Comparative Example 2 (C2)

205.6 g (24 mmol) of PEG 8000 (OH number=13.1) were introduced as initial charge in the 1 l four-neck flask. It was evacuated twice and aerated with nitrogen. A vacuum was then applied and the mixture was heated to 100° C. At this temperature and under a reduced pressure of at least 10 mbar, water was removed over a period of 2 hours. The system was then aerated with nitrogen and, in the further course, the protective-gas atmosphere was maintained by a gentle stream of nitrogen. The mixture was stirred over the entire following reaction time using a stirrer speed of 120 rpm. Then, in succession, 10.1 g (69.9 mmol) of nonanol and 15.5 g of isophorone diisocyanate (69.9 mmol) were added. The reaction temperature was held at 110° C. during the addition and during the subsequent reaction time. As soon as residual isocyanate could no longer be detected (this was the case after a total reaction time of ca. 45 minutes), the temperature was kept at 110° C. and 839.6 g of demin. water were added. This mixture was stirred until homogeneous. After the product had cooled down, ca. 1003.7 g of a viscous, clear, slightly yellowish polymer solution were isolated from the reaction vessel.

The dry residue (for its determination ca. 2 g of the polymer solution prepared as described above were dried in a 10 cm aluminum dish at 105° C. for 1.5 h in a convection drying cabinet) was 18.3% by weight and the Brookfield viscosity was 2250 mPas (Brookfield RVT viscometer/spindle 3/10 rpm/22° C.)

The coating viscosity in the Epprecht viscometer was 120 mPas (spindle C).

### Comparative Example 3 (C3)

185.7 g (24 mmol) of PEG 8000 (OH number=14.5) were introduced as initial charge in the 1 l four-neck flask. It was evacuated twice and aerated with nitrogen. A vacuum was then applied and the mixture was heated to 100° C. At this temperature and under a reduced pressure of at least 10 mbar, water was removed over a period of 2 hours. The system was then aerated with nitrogen and, in the further course, the protective-gas atmosphere was maintained by a gentle stream of nitrogen. The mixture was stirred over the entire following reaction time using a stirrer speed of 120 rpm. Then, in succession, 12.0 g of Exxal 13 (60 mmol) and 16.0 g of isophorone diisocyanate (72 mmol) were added. The reaction temperature was held at 110° C. during the addition and during the subsequent reaction time. As soon as residual isocyanate could no longer be detected (this was the case after a total reaction time of ca. 60 minutes), the temperature was kept at 110° C. and 127.2 g of DEHYDOL® O4 were added. This mixture was stirred until homogeneous. Then, 300.7 g of demin. water were added with stirring. After the product had cooled down, ca. 635.8 g of a viscous, clear, slightly yellowish polymer solution were isolated from the reaction vessel.

The dry residue (for its determination ca. 2 g of the polymer solution prepared as described above were dried in a 10 cm aluminum dish at 105° C. for 1.5 h in a convection drying cabinet) was 49.3% by weight and the Brookfield viscosity was 10 600 mPas (Brookfield RVT viscometer/spindle 3/10 rpm/23° C.)

The coating viscosity in the Epprecht viscometer was 220 mPas (spindle C).

### Determination of the Dispersion Thickening

Thickening Effectiveness:

0.2 g of the polymer solution obtained according to example 1 was homogenized with 19.8 g of a mixture JY 844 (the mixture JY 844 had the following composition: NEOCRYL® XK 98 (94.8 parts by weight), ammonia 25% (0.2 part by weight), butyl diglycol (2.3 parts by weight), DEHYDRAN® 1513 (1.0 part by weight)). The resulting composition was placed in a convection cabinet for 60 min at 45° C. and was homogenized by shaking several times. After a standing time of 20 hours at 25° C., it was shaken again.

In a completely analogous manner, the polymer solutions obtained according to the other examples (E2 and E3) and comparative examples (C1, C2 and C3) were investigated as to their thickening effectiveness. The results of all of the experiments are shown in table 1 below.

TABLE 1

| Polymer | | | Dispersion viscosity [mPas] | | |
|---|---|---|---|---|---|
| solution | | | JY-844 | | |
| according | | Product | | | ICI |
| to | Dry | viscosity | | | Cone C, |
| example | residue | [mPas] | 0.5 s⁻¹ | 500 s⁻¹ | 25° C./10 000 |
| E1 | 51.3% | 5600 | 6.723 | 1.408 | 220 |
| E2 | 18.3% | 7650 | n.d. | 0.745 | 150 |
| E3 | 51.1% | 8700 | 35.084 | 2.501 | 450 |
| C1 | 50.9% | 4850 | 4.671 | 1.321 | 200 |
| C2 | 18.3% | 2250 | n.d. | 0.511 | 120 |
| C3 | 49.3% | 10 600 | 34.169 | 2.323 | 220 |

What is claimed is:

1. A polyurethane obtained by reacting:
(i) one or more hydrophilic polyols which contain, per molecule, at least two OH groups and at least two functional groups which are selected from the functions —O— (ether) and —COO— (ester), provided that the molecular weight of said hydrophilic polyols is at least 300 Daltons;
(ii) one or more monoalcohols having in total 8 to 36 carbon atoms, with the proviso that said mono-alcohols are Guerbet alcohols, which are obtained by using as starting alcohols for a Guerbet reaction, the primary and/or secondary monoalcohols of the formula (MA), 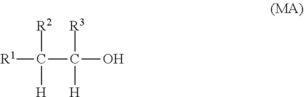
wherein (a) the total number of carbon atoms in the compounds (MA) is in the range from 4 to 18; (b) R¹, R² and R³ are independently hydrogen, or linear, branched or alicyclic, saturated or unsaturated alkyl; (c) optionally, R¹ and R², and/or R¹ and R³, and/or R² and R³ are linked together as part of an alicyclic substructure; and (d) the compounds (MA) contain at least one branch;
and
(iii) one or more at least difunctional isocyanates.

2. The polyurethane of claim 1, wherein said polyols (i) are selected from the group consisting of polyethylene glycols and polypropylene glycols.

3. The polyurethane of claim 1, wherein said monoalcohols (MA) comprise saturated and at least monobranched monoalcohols having 5 to 10 carbon atoms.

4. The polyurethane of claim 1, wherein said monoalcohol (MA) comprises 3-methylbutan-1-ol.

5. The polyurethane of claim 1, wherein said compounds (i), (ii) and (iii) are reacted together in the equivalent ratios OHᵢ₎:ZHᵢᵢ₎:NCOᵢᵢᵢ₎ of 1:(1+x):2(1+y), provided that x is a number in the range from 0.05 to 1.2, y is a number in the range from (0.2 to 1.05)x, and the equivalent ratio NCOᵢᵢᵢ₎ is greater than the sum (OHᵢ₎+ZHᵢᵢ₎).

6. The polyurethane of claim 1, wherein said isocyanate (iii) is selected from the group consisting of isophorone diisocyanate and tetramethylxylene diisocyanate.

7. The polyurethane of claim 1, wherein said polyurethane is obtained by reacting said compounds (i), (ii), (iii), and further, compound (iv), which comprises one or more polyols having at least two OH groups per molecule, and which also, apart from said OH groups, contains no further functional groups.

8. The polyurethane of claim 7, wherein said polyol (iv) comprises trimethylolpropane.

9. The polyurethane of claim 7, wherein said polyurethane is obtained by reacting said compounds (i), (ii), (iii), (iv), and further, compound (v), which comprises one or more monoalcohols which are different from the compounds (ii).

10. The polyurethane of claim 9, wherein said monoalcohols (v) are selected from the group consisting of Guerbet alcohols having 8 to 24 carbon atoms which have at most two branches per molecule, and oxo alcohols.

11. A thickener concentrate comprising one or more polyurethanes of claim 1, and water.

12. The thickener concentrate of claim 11, wherein said concentrate further comprises one or more organic solvents, and/or one or more nonionic surfactants, which surfactants comprise the addition compounds of ethylene oxide and/or propylene oxide onto fatty alcohols having 4 to 18 carbon atoms.

13. A method for thickening emulsion paints, comprising the step of adding the thickener concentrate of claim 11 to a paint base.

14. A method for thickening aqueous dispersions, comprising the step of adding the thickener concentrate of claim 11 to an aqueous dispersion.

15. The method of claim 14, wherein said aqueous dispersion comprises a cosmetic preparation.
